(19) United States
(12) Patent Application Publication
(10) Pub. No.: US 2002/0187080 A1
(43) Pub. Date: Dec. 12, 2002

(54) Containment assembly

(76) Inventors: Michael Mellor, Brighouse (GB); Neil Cocker, Penistone (GB)

Correspondence Address: Donald J. Harrington, Brooks & Kushman P.C., 22nd Floor, 1000 Town Center, Southfield, MI, 48075-1351, US

(21) Appl. No.: 10/161,928

(22) Filed: Jun. 4, 2002

(30) Foreign Application Priority Data
Jun. 5, 2001 (GB) 0113506.0

(51) Int. Cl. A61L 9/00

(52) U.S. Cl. 422/120; 588/249

(57) ABSTRACT

A containment assembly with gloved access to a processing zone that is isolated from the surrounding environment. A protective glove is secured in an access opening. A replacement glove is secured in a retainer location that is separate from the retainer location of the glove being replaced without interrupting the isolation of the processing zone.

### CROSS-REFERENCE TO RELATED APPLICATIONS

This application claims priority to Great Britain Patent Application No. 0113506.0, filed Jun. 5, 2001.

### BACKGROUND OF THE INVENTION

1. Field of the Invention

The present invention relates to a containment assembly with improved gloved access to a processing zone.

2. Background Art

Effective containment is essential for the sale and hygienic handling of a pharmaceutical, chemical or biological product. At each stage, handling must be controlled and managed to provide optimum protection for the operator and for maintaining the integrity of the product. At the conclusion of each processing operation, the interior surfaces of the structure enclosing the processing zone must be thoroughly cleaned by the operator prior to a further processing operation to minimize the risk of cross-contamination. Current good manufacturing practices exercised within the pharmaceutical manufacturing industry demand good containment performance and acceptable operator exposure levels.

For handling a solid (e.g., powder) or liquid product of a sensitive or hazardous (e.g., toxic) nature in a processing zone, there are available a number of different types of containment assembly. One such conventional containment assembly is a barrier isolator with gloved access to the processing zone in which a product and/or handling equipment may be manipulated. A barrier isolator may offer two fold protection, namely the use of glove ports to maintain a physical barrier between the product and the operator and a fan system to create air flow for removing airborne particles from the processing zone and into filters. In this manner, a barrier isolator can achieve high containment, typically down to nanogramme level. Frequently, a barrier isolator is adapted to provide net inward air flow (i.e., it exhibits a negative pressure relative to the environment). This lessens the risk of airborne particles entering the environment, in particular in the event of glove failure.

### SUMMARY OF THE INVENTION

The present invention is based on the recognition that eliminating the void between the glove and the interior surface of the glove port improves the performance of a containment assembly by reducing the risk of cross-contamination. More particularly, the present invention relates to a containment assembly adapted to confine the portion of the glove adjacent to the interior surface of the glove port so as to lessen the risk of debris being trapped.

Thus viewed from one aspect, the present invention provides a containment assembly (e.g., an isolator) comprising a walled housing erected so as to enclose a processing zone beyond an access wall thereof; a substantially circular aperture within the access wall; a substantially cylindrical glove port fitted to the access wall at or near to the circumference of and coaxial with the substantially circular aperture, an exterior surface of which extends into a retaining portion at the front of the access wall; a flexible glove retained on the retaining portion of the glove port, said flexible glove being adapted or adaptable to permit an operator outside the processing zone to reach through the substantially circular aperture of the access wall into the processing zone; and a confinement member positioned so as to confine a part of the flexible glove against an interior surface of the substantially cylindrical glove port.

Preferably, the walled housing is faceted with the substantially circular aperture within one face thereof. The walled housing may be multi-faceted with the substantially circular aperture within one face thereof. Typically, the walled housing adopts a substantially cuboidal configuration (e.g., a box-like configuration) having a rear wall, a front wall, a basal wall, a first side wall, a second side wall and an upper wall, wherein the front wall is the access wall.

The substantially circular aperture within the access wall of the walled housing may be circular, elliptical or oval as desired. However, it is not intended to exclude other shapes (e.g., curved, partially curved or non-curved shapes) from the scope of the invention. It will be appreciated that the substantially cylindrical glove port may be shaped to conform to the shape of the substantially circular aperture so as to be fitted to the access wall at or near to the circumference thereof and coaxial therewith.

In an embodiment of the invention, the confinement member is a substantially cylindrical confinement member at least partially inserted coaxially into the substantially cylindrical glove port so as to confine a part of the flexible glove against the interior surface of the substantially cylindrical glove port.

In an embodiment of the invention, the confinement member extends radially and outwardly into a flange adapted to be secured to the front of the access wall. For example, the substantially cylindrical confinement member extends radially and outwardly into an annular flange adapted to be secured to the front of the access wall. The flange may have a bore (e.g., a countersunk bore) for receiving a fastener (e.g., a threaded fastener such as a screw), which may secure the flange to the front of the access wall directly or indirectly.

The annular flange may incorporate an annular recess encapsulating the retaining portion of the substantially cylindrical glove port. By protectively encapsulating the retaining portion, an embodiment of the invention has the added advantage that the risk of glove failure or of accidental dislodgement of the flexible glove is reduced.

In an embodiment of the invention, the substantially cylindrical glove port is fitted to the front face of the access wall at or near to the circumference of and coaxial with the substantially circular aperture.

The substantially cylindrical glove port may be fitted at or near to the edge of the access wall bounding the substantially circular aperture. Preferably the substantially cylindrical glove port is fitted near to the edge of the access wall bounding the substantially circular aperture (i.e., there is a narrow gap between the edge of the access wall bounding the substantially circular aperture and the substantially cylindrical glove port).

The substantially cylindrical glove port may be fitted to the front face of the access wall or at or near to the edge of the access wall bounding the substantially circular aperture in any convenient manner such as by using a mechanical attachment and/or an adhesive (e.g., using conventional adhesives and/or fasteners such as threaded fasteners; for example, screws).

In this regard, the containment assembly preferably further comprises a clamping ring bearing against the front face of the access wall, the clamping ring being adapted to secure the substantially cylindrical glove port at or near to the edge of the access wall bounding the substantially circular aperture. For example, the clamping ring may incorporate a first bore capable of receiving a fastener (e.g., a threaded fastener), said first bore being collinear with a receiving bore in the substantially cylindrical glove port, said receiving bore being capable of receiving the fastener (e.g., the threaded fastener). For this purpose, the substantially cylindrical glove port may extend radially and outwardly into a flange bearing against the rear face of the access wall. In an embodiment of the invention, the clamping ring may incorporate a second bore capable of receiving a second fastener (e.g., a second threaded fastener), said second bore being collinear with the bore in the flange extending radially and outwardly from the confinement member (as described in the embodiment hereinbefore). In this manner, the flange may be indirectly secured to the front of the access wall.

The exterior surface of the substantially cylindrical glove port may extend radially or axially into the retaining portion. Preferably the exterior surface of the substantially cylindrical glove port extends axially away from the front of the access wall to define a retaining portion incorporating a first retaining groove and a second retaining groove. The first and/or second retaining groove is typically an annular retaining groove. Preferably the first retaining groove is disposed forwardly of the second retaining groove and is generally shallower than the second retaining groove. The first retaining groove may define a substantially square cross-section. The first retaining groove is generally used to retain the flexible glove during normal operation of the containment assembly. The second retaining groove may define a curved (e.g., substantially hemispherical) cross-section. The second retaining groove is generally used to retain a replacement flexible glove during a glove change operation.

Generally speaking, the flexible glove is composed of natural rubber (latex), Hypalon, neoprene, butyl or polyurethane rubbers. The flexible glove may be retained in the retaining portion in any conventional manner. For example, the rolled edge of the flexible glove may be sufficient to retain it in the retaining portion. A strengthening ring (e.g., an o-ring) may be used to positively retain the flexible glove in the retaining portion.

In an embodiment of the invention, the flexible glove comprises a flexible sleeve terminating in a gloved end. Preferably, in use the flexible sleeve extends beyond the operator's elbow to optimize manoeuverability. The gloved end is typically composed of less flexible material than the flexible sleeve. For example, the gloved end may be composed of thicker polyurethane than that of the flexible sleeve (or alternatively of thicker PVC or other material).

The gloved end may be attached to the flexible sleeve in any conventional manner. Preferably, the joint between the gloved end and the flexible sleeve comprises an internal strengthening ring to which the ends of the gloved end and the flexible sleeve are secured. For example, the ends of the gloved end and the flexible sleeve may be secured to the internal strengthening ring by an elastic band or the like.

Typically, the position and orientation of the flexible glove may be tailored to suit the particular operation to be conducted in the processing zone. For example, the flexible glove may be right-hand orientated or left-hand orientated as desired.

Where appropriate, a plurality of substantially circular apertures may be provided within the access wall of the walled housing. One or more additional walls of the walled housing may be provided with one or more substantially circular apertures. Each of these may be separately provided with a glove port and flexible glove in accordance with the invention.

In a preferred embodiment, the confinement member (e.g., the substantially cylindrical confinement member) sealingly confines the part of the flexible glove against the interior surface of the substantially cylindrical glove port.

For this purpose, the interior surface of the substantially cylindrical glove port advantageously extends radially and inwardly into a retaining shoulder, the retaining shoulder being in substantially sealing engagement with the end of the confinement member. The retaining shoulder may be disposed at or near to the end of the interior surface of the substantially cylindrical glove port. Preferably the retaining shoulder is an annular retaining shoulder. Preferably, the retaining shoulder is of a substantially triangular cross-section and is in sealing engagement with the end of the confinement member. For this purpose, the end of the confinement member may be chamfered to mate with the retaining shoulder and improve the sealing engagement.

The air quality in and around the processing zone may be controlled using forced ventilation. For example, the containment assembly may be adapted to provide non-circulatory air flow in the processing zone. For this purpose, air exhausted from the processing zone may be ducted to atmosphere or to a remote location. Alternatively, the containment assembly may be adapted to provide recirculatory air flow in the processing zone (e.g., “down flow” in which surrounding air is pushed downwardly to entrain airborne particles to a level below the process zone; e.g., to a level below the typical operator's breathing zone). For down flow containment, exhaust is usually carried out at low level relative to the source of particles.

The containment assembly of the invention may be any of the types that use gloved access, including isolators, fume cupboards and filter scan boxes. The versatility of the containment assembly of the invention is such that it may be used in numerous applications including the containment of chemical, pharmaceutical or biological (e.g., microbiological) powders or liquids.

### BRIEF DESCRIPTION OF THE DRAWINGS

FIGS. 1(a) to 1(d) show a conventional containment assembly for isolating a processing chamber; and

FIGS. 2(a) to 2(e) are partial cross-sectional views of the containment assembly of the invention in an assembled state and in a partially disassembled state.

### DETAILED DESCRIPTION OF THE PREFERRED EMBODIMENT(S)

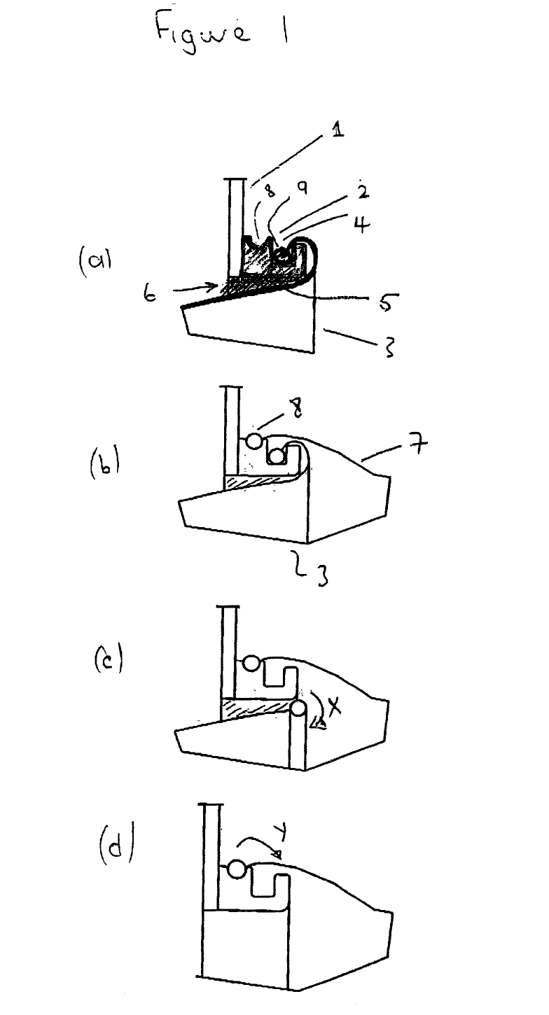

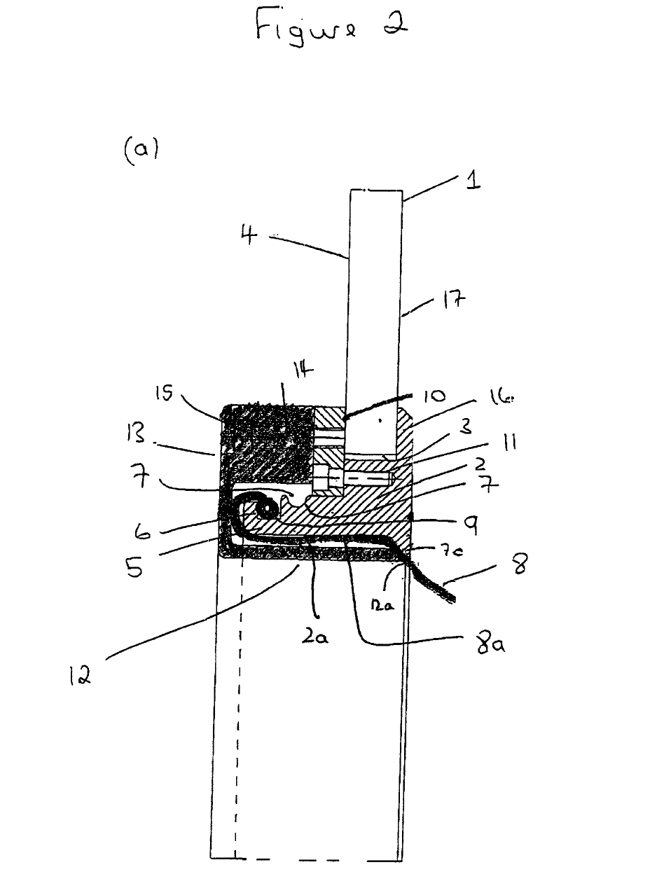

Gloved access to the processing zone in a barrier isolator of this type is typically achieved in the manner illustrated schematically in partial cross-sectional views in FIGS. 1(a)-1(d). An access wall 1 of the isolator contains a substantially circular aperture and is fitted with a glove port 2 (e.g., by an adhesive) near to the circumference of the aperture. The glove port 2 comprises a lower retaining groove 4 and upper retaining groove 8. In the normal working condition, a glove 3 is retained in the lower retaining groove 4 by virtue of a strengthening ring 9. Between the interior surface 5 of the glove port 2 and the glove 3 is a void 6. The void 6 is more pronounced when the isolator is operated under negative pressure.

During normal operation or during the cleaning cycle, debris from a solid (e.g., powder) or liquid product or cleaning agents may become trapped in the void 6. Since the void 6 is to all intents and purposes inaccessible by the operator, it cannot be effectively cleaned and therefore seriously compromises the containment performance of the isolator. Moreover, it will be seen from FIGS. 1(b) to 1(d) that the glove change procedure serves to withdraw the trapped debris into the processing zone. A replacement glove 7 is fitted in the upper retaining groove 8 over the existing glove 3. The existing glove 3 is removed from the lower retaining groove 4 (see X), pushed through the glove port 2 and the aperture in the access wall 1 and removed from the processing zone via the normal exit. The replacement glove 7 may then be re-positioned into the lower retaining groove 4 ready for use (see Y).

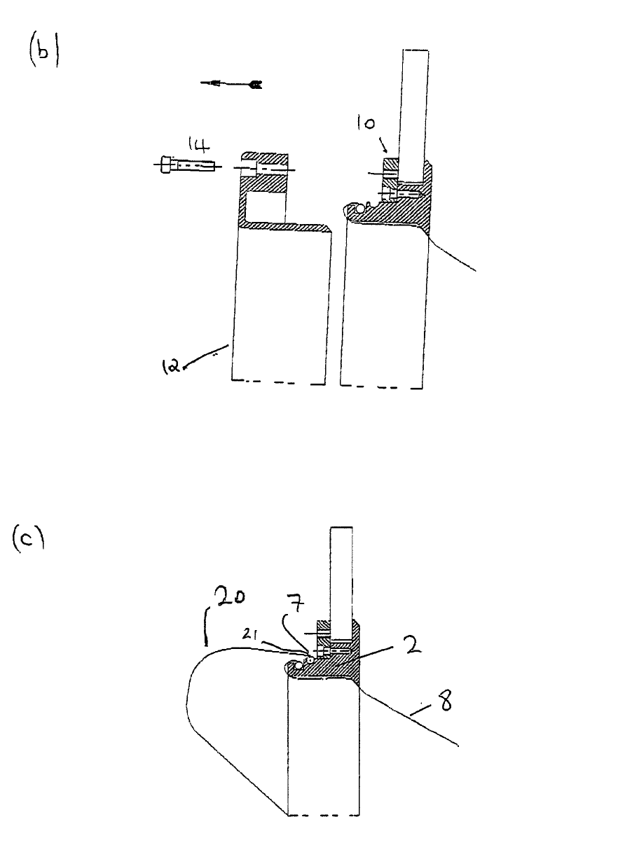

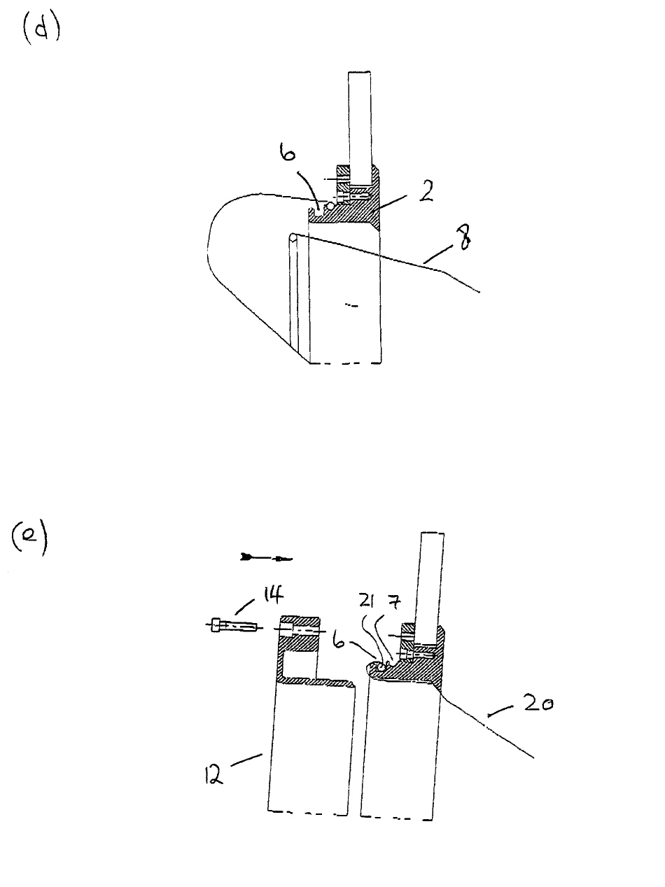

The present invention will now be described in a non-limitative sense with reference to accompanying FIG. 2 in which FIGS. 2(a)-2(e) illustrate an embodiment of the containment assembly of the invention and the safe change procedure.

In the embodiment illustrated schematically in partial cross-sectional view in FIGS. 2(a)-2(e), an access wall 1 of a walled housing contains a substantially circular aperture. Near to an edge 3 of the access wall 1 bounding the aperture is fitted a substantially cylindrical glove port 2. The exterior surface of the substantially cylindrical glove port 2 extends axially into a retaining portion 5 at the front face 4 of the access wall 1. The retaining portion 5 incorporates a first annular retaining groove 6 of square cross-section and a second annular retaining groove 7 of curved cross-section. In the normal working condition illustrated in FIG. 2(a), a glove 8 is fitted in the first annular retaining groove 6 by the action of a strengthening o-ring 9.

The substantially cylindrical glove port 2 extends radially and outwardly into a flange 16 bearing against the rear face 17 of the access wall 1. The substantially cylindrical glove port 2 is secured near to the edge 3 of the access wall bounding the aperture by a clamping ring 10 bearing against the front face 4 of the access wall 1. A threaded fastener 11 passes through collinear, threaded bores in the clamping ring 10 and substantially cylindrical glove port 2 to secure the glove port 2 in place.

A substantially cylindrical confinement member 12 is coaxially inserted into the substantially cylindrical glove port 2 so as to confine a part of the flexible glove 8a against the interior surface 2a of the substantially cylindrical glove port 2. A seal is effectively formed between the end 12a of the substantially cylindrical confinement member 12 and an annular shoulder 70 extending radially inwardly from the interior surface 2a of the substantially cylindrical glove port 2.

The substantially cylindrical confinement member 12 extends radially outwardly into an annular flange 13 encapsulating the retaining portion 5 of the substantially cylindrical glove port 2. The annular flange 13 is provided with a threaded bore 14 which receives a threaded fastener 15 for securing the confinement member 12 to the clamping ring 10 (and, therefore, indirectly to the front face of the access wall 3).

FIGS. 2(b)-2(e) illustrate schematically the glove change procedure. In the first step (FIG. 2(b)), the substantially cylindrical confinement member 12 is removed by releasing the threaded fastener 14 to expose the clamping ring 10. In the second step (FIG. 2(c)), a strengthening ring 21 of a replacement glove 20 is fitted in the second annular retaining groove 7 of the substantially cylindrical glove portion 2 over the old glove 8. In the next step (FIG. 2(d)), the old glove 8 is dislodged from the first annular retaining groove 6 and pushed through the glove port 2 and removed from the processing zone via the normal exit. In the next step, the glove ring 21 of the glove 20 is dislodged from the second annular retaining groove 7 and inserted into the first annular retaining groove 6. The confinement member 12 is repositioned and re-fastened using threaded fastener 14 leaving the glove ready for use.

While embodiments of the invention have been illustrated and described, it is not intended that these embodiments illustrate and describe all possible forms of the invention. Rather, the words used in the specification are words of description rather than limitation, and it is understood that various changes may be made without departing from the spirit and scope of the invention.

What is claimed is:

1. A containment assembly comprising:
a walled housing erected so as to enclose a processing zone beyond an access wall thereof;
a substantially circular aperture within the access wall;
a substantially cylindrical glove port fitted to the access wall at or near to the circumference of and coaxial with the substantially circular aperture, an exterior surface of which extends into a retaining portion at the front of the access wall;
a flexible glove retained on the retaining portion of the glove port, said flexible glove being adapted or adaptable to permit an operator outside the processing zone to reach through the substantially circular aperture of the access wall into the processing zone; and
a confinement member positioned so as to confine a part of the flexible glove against an interior surface of the substantially cylindrical glove port.

2. A containment assembly as claimed in claim 1 wherein the walled housing is faceted with the substantially circular aperture within one face thereof.

3. A containment assembly as claimed in claim 2 wherein the walled housing is multi-faceted with the substantially circular aperture within one face thereof

4. A containment assembly as claimed in claim 3 wherein the walled housing adopts a substantially cuboidal configuration having a rear wall, a front wall, a basal wall, a first side wall, a second side wall and an upper wall, wherein the front wall is the access wall.

5. A containment assembly as claimed in claim 1 wherein the confinement member is a substantially cylindrical confinement member as least partially, coaxially inserted into the substantially cylindrical glove port so as to confine a part of the flexible glove against the interior surface of the substantially cylindrical glove port.

6. A containment assembly as claimed in claim 5 wherein the confinement member extends radially and outwardly into a flange adapted to be secured to the front of the access wall.

7. A containment assembly as claimed in claim 5 wherein the substantially cylindrical confinement member extends radially and outwardly into an annular flange adapted to be secured to the front of the access wall.

8. A containment assembly as claimed in claim 6 wherein the flange has a bore for receiving a fastener for securing the flange to the front of the access wall directly or indirectly.

9. A containment assembly as claimed in claim 7 wherein the annular flange incorporates an annular recess encapsulating the retaining portion of the substantially cylindrical glove port.

10. A containment assembly as claimed in claim 6 wherein the substantially cylindrical glove port is fitted at or near to the edge of the access wall bounding the substantially circular aperture.

11. A containment assembly as claimed in claim 7 wherein the substantially cylindrical glove port is fitted n ear to the edge of the access wall bounding the substantially circular aperture.

12. A containment assembly comprising:
a walled housing erected so as to enclose a processing zone beyond an access wall thereof;
a substantially circular aperture within the access wall;
a substantially cylindrical glove port fitted to the access wall at or near to the circumference of and coaxial with the substantially circular aperture, an exterior surface of which extends into a retaining portion at the front of the access wall;
a flexible glove retained on the retaining portion of the glove port, said flexible glove being adapted or adaptable to permit an operator outside the processing zone to reach through the substantially circular aperture of the access wall into the processing zone;
a confinement member positioned so as to confine a part of the flexible glove against an interior surface of the substantially cylindrical glove port; and
a clamping ring bearing against the front face of the access wall, said clamping ring being adapted to secure the substantially cylindrical glove port at or near to the edge of the access wall bounding the substantially circular aperture.

13. A containment assembly as claimed in claim 12 wherein the clamping ring incorporates a first bore capable of receiving a fastener, said first bore being collinear with a receiving bore in the substantially cylindrical glove port, said receiving bore being capable of receiving the fastener.

14. A containment assembly as claimed in claim 13 wherein the substantially cylindrical glove port extends radially and outwardly into a flange bearing against the rear face of the access wall.

15. A containment assembly as claimed in claim 12 wherein the clamping ring incorporates a second bore capable of receiving a second fastener, said second bore being collinear with a bore in a flange extending radially and outwardly from the confinement member.

16. A containment assembly as claimed in claim 13 wherein the exterior surface of the substantially cylindrical glove port extends radially or axially into the retaining portion.

17. A containment assembly as claimed in claim 16 wherein the exterior surface of the substantially cylindrical glove port extends axially a way from the front of the access wall to define a retaining portion incorporating a first retaining groove and a second retaining groove.

18. A containment assembly as claimed in claim 17 wherein the first and/or second retaining groove is an annular retaining groove.

19. A containment assembly as claimed in claim 17 wherein the first retaining groove is disposed forwardly of the second retaining groove and is generally shallower than the second retaining groove.

20. A containment assembly as claimed in claim 17 wherein the first retaining groove defines a substantially square cross-section.

21. A containment assembly as claimed in claim 17 wherein the second retaining groove defines a curved cross-section.

22. A containment assembly as claimed in claim 17 wherein the second retaining groove defines a substantially hemispherical cross-section.

23. A containment assembly as claimed in claim 17 wherein the confinement member sealingly confines the part of the flexible glove against the interior surface of the substantially cylindrical glove port.

24. A containment assembly as claimed in claim 23 wherein the interior surface of the substantially cylindrical glove port extends radially and inwardly into a retaining shoulder, said retaining shoulder being a substantially sealing engagement with the end of the confinement member.

25. A containment assembly as claimed in claim 24 wherein the retaining shoulder is disposed at or near to the end of the interior surface of the substantially cylindrical glove port.

26. A containment assembly as claimed in claim 24 wherein the retaining shoulder is an annular retaining shoulder.

27. A containment assembly as claimed in claim 24 wherein the retaining shoulder is of a substantially triangular cross-section and is in sealing engagement with the end of the confinement member.

28. A containment assembly as claimed in claim 24 wherein the end of the confinement member is chamfered to mate with the retaining shoulder.
